(19) United States
(12) Patent Application Publication
(10) Pub. No.: US 2025/0340811 A1
(43) Pub. Date: Nov. 6, 2025

(54) APPARATUS FOR TRANSPORTING AND INSTALLING A BIOPROCESSING VESSEL

(71) Applicant: Global Life Sciences Solutions USA LLC, Marlborough, MA (US)

(72) Inventors: Kevin Ashley Lamport, Portsmouth Hampshire (GB); Matthew Ouellette, Marlborough, MA (US); Boudewijn van Limpt, Medemblik (NL)

(73) Assignee: Global Life Sciences Solutions USA LLC, Marlborough, MA (US)

(21) Appl. No.: 18/654,163

(22) Filed: May 3, 2024

(51) Int. Cl. C12M 1/00; B65D 19/00; B65D 19/42

(52) U.S. Cl. CPC C12M 23/52; B65D 19/0002; B65D 19/42; C12M 23/26; C12M 23/40; C12M 29/16

(57) ABSTRACT

An apparatus for transporting and installing a flexible bioprocessing vessel includes a base portion for attachment to a bottom panel of the vessel, the base portion having first and second wing portions selectively movable relative to the base portion between first and second positions. The apparatus includes a top plate for attachment to a top panel of the vessel. The apparatus includes a front panel connected to a front end of the base portion, the front panel including a plurality of apertures configured to secure and protect tubing and/or connectors extending from the vessel. In the first position, the first and second wing portions are operatively connected to first and second sides of the top plate forming a storage cavity for the vessel. In the second position, the first and second wing portions are disconnected from the first and second sides of the top plate.

### BACKGROUND

### Technical Field

Embodiments of the invention relate generally to bioprocessing, and, more particularly, to an apparatus for transporting and installing a flexible bioprocessing vessel within a rigid support structure.

### Discussion Of Art

Mixers and bioreactors are often employed to carry out biochemical and biological processes and/or manipulate liquids and other products of such processes. These devices typically utilize single-use vessels e.g., flexible or collapsible bags that are supported by an outer rigid structure such as a stainless-steel housing/tank.

In use, a disposable/single-use bag is positioned within the rigid tank and filled with the desired fluid for processing. An impeller assembly that includes a rotating impeller having one or more blades is disposed within the bag and is used to mix the fluid. Existing impeller systems are either top-driven, having a shaft that extends downwardly into the bag, on which one or more impellers are mounted, or bottom-driven, having an impeller disposed in the bottom of the bag that is driven by a drive system positioned outside the bag. Such drive systems may be direct drive or magnetic.

Rigid support structures, e.g., stainless steel tanks, may be relatively large, having capacities of 500 L, 2000 L, and 3000 L or more. As will be appreciated, flexible bags configured for use within such support structures are likewise relatively large and given their size and flexible nature, may be challenging to transport and install within such structures. Packaging solutions have been developed to assist in the transport of certain bags. These generally include a base portion that supports the flexible bag during transport and include handles that allow users to lift the bag out of its external shipping container, e.g., a cardboard box, and place the bag in a tank for use. Existing packaging, however, does not include tubing management solutions that protect tubing and connectors during transport, facilitate the connection of tubing, and deployment of the vessel within a rigid support structure.

Moreover, when a flexible vessel/bag is being raised into position within a rigid support structure, the film of the bag may contact the interior of the structure and may stick to an interior surface. Current packaging solutions do not address this potential issue.

In addition, for large vessels/bags a hoist or lift mechanism may be utilized to raise the bag within the tank to a fully deployed position where it can be used. Known packaging solutions do not assist in this process, i.e., they do not include an upper plate or other surface suitable for lifting that remains attached during use of the bag.

It may also be desirable to utilize a flexible vessel/bag that has a hexagonal structure, e.g., a bag having eight flexible panels (six side panels, a top panel, and a bottom panel, that are welded together) within a cylindrical rigid support structure, e.g., tank. There are currently no packaging solutions for the transport, installation, and deployment of such a vessel in a cylindrical tank.

In view of the above, there is a need for an apparatus for transporting and installing a flexible bioprocessing vessel in a rigid cylindrical support structure. There's also a need for a transport apparatus provides tubing/connector protection and organization, as well as protection to the vessel/bag as its being raised/deployed in a tank.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of the possible embodiments. Indeed, the disclosure may encompass a variety of forms that may be similar to or different from the embodiments set forth below.

According to an aspect of the invention, an apparatus for transporting and installing a flexible bioprocessing vessel includes a base portion configured for operative connection to a bottom panel of the flexible bioprocessing vessel, the base portion having a first wing portion and a second wing portion that are selectively movable relative to the base portion between a first position and a second position. The apparatus further includes a top plate configured for operative connection to a top panel of the flexible bioprocessing vessel, the top plate remaining attached to the flexible bioprocessing vessel during use. The apparatus includes a front panel connected to a front end of the base portion, the front panel including a plurality of apertures configured to secure and protect tubing and/or connectors extending from the flexible bioprocessing vessel during transportation and/or use. Wherein in the first position, the first wing portion and the second wing portion are operatively connected to a first side and a second side of the top plate forming a storage cavity for the flexible bioprocessing vessel that facilitates transportation and/or installation of the flexible bioprocessing vessel. Wherein in the second position, the first wing portion and the second wing portion are disconnected from the first side and second side of the top plate facilitating installation and/or use of the flexible bioprocessing vessel within a rigid support structure.

In an embodiment, the apparatus may include a selectively removable support base configured for connection to an exterior of the base portion, the selectively removable support base configured to protect portions of the flexible bioprocessing vessel that extend from the apparatus during transportation of the flexible bioprocessing vessel.

In an embodiment, a front surface and/or a rear surface of the top plate includes at least one slot, aperture, and/or opening configured to receive and/or support tubing of the flexible bioprocessing vessel.

In an embodiment, the first wing portion and the second wing portion each have at least one handle.

In an embodiment, the base portion may be substantially circular and configured for placement within a cylindrical rigid support structure.

In an embodiment, the front panel has a curved profile.

In an embodiment, the top plate includes two side flaps that are connected to the first wing portion and second wing portion of the base portion in the first position.

In an embodiment, the apparatus of claim 1, may be configured for use with a 2000 L flexible bioprocessing vessel.

In an embodiment, the first wing portion and the second wing portion are connected to the first side and the second side of the top plate via removable side panels.

In an embodiment, the removable side panels are secured to the first wing portion and/or the second wing portion by one or more cable ties.

In an embodiment, each of the removable side panels include one or more handles.

In an embodiment, the top plate is configured to receive tubing of the flexible bioprocessing vessel during transportation.

In an embodiment, the apparatus includes a removable beam configured to secure the tubing of the flexible bioprocessing vessel to the top plate.

In an embodiment, the flexible bioprocessing vessel may include at least 8 panels that are secured together to form an interior configured to receive fluid for bioprocessing.

In another aspect of the invention, a system for transporting and installing a flexible bioprocessing vessel includes a flexible bioprocessing vessel having a body that includes a plurality of panels that are secured together to form an interior cavity configured to receive fluids for bioprocessing, the body having a bottom panel and a top panel. The system further includes a base portion operatively connected to the bottom panel of the flexible bioprocessing vessel and a top plate operatively connected to the top panel of the flexible bioprocessing vessel, the top plate and base portion remaining attached to the flexible bioprocessing vessel during transportation and/or use. The system also includes a front panel connected to a front end of the base portion, the front panel including a plurality of apertures configured to secure and protect tubing and/or connectors extending from the flexible bioprocessing vessel during transportation and/or use. Wherein in a first position, the base portion is operatively connected to the top plate forming a storage cavity for the flexible bioprocessing vessel facilitating transportation of the flexible bioprocessing vessel. Wherein in a second position, the base portion and the top plate are disconnected from each other facilitating installation and use of the flexible bioprocessing vessel within a rigid support structure.

In an embodiment, the system further includes a selectively removable support base configured for connection to an exterior of the base portion, the selectively removable support base configured to protect portions of the flexible bioprocessing vessel that extend from the base portion during transportation of the flexible bioprocessing vessel.

In an embodiment, a front surface and/or a rear surface of the top plate includes at least one slot configured to receive and support tubing facilitating use of the flexible bioprocessing vessel.

In an embodiment, the base portion includes at least one handle.

In an embodiment, the base portion is substantially circular and configured for placement within a cylindrical rigid support structure.

In an embodiment, the flexible bioprocessing vessel may include at least 8 panels that are secured together.

In yet another aspect, a method for transporting and installing a flexible bioprocessing vessel includes operatively connecting a base portion of a support apparatus to a bottom panel of the flexible bioprocessing vessel. The method further includes operatively connecting a top plate of the support apparatus to a top panel of the flexible bioprocessing vessel, the base portion and top plate remaining attached to the flexible bioprocessing vessel after installation within a rigid support structure and attaching the top plate to the base portion to form a cavity for the flexible bioprocessing vessel. The method also includes transporting the flexible bioprocessing vessel to a desired location and installing the flexible bioprocessing vessel with a rigid support structure.

In an embodiment, installing the flexible bioprocessing vessel includes placing the support apparatus within a rigid support structure, detaching the top plate from the base portion, deploying the flexible bioprocessing vessel.

In an embodiment, the method further includes securing a selectively removable support base to an exterior of the base portion.

In an embodiment, the method further includes securing tubing of the flexible bioprocessing vessel within apertures in a front panel of the base portion of the support apparatus prior to transporting the flexible bioprocessing vessel.

### BRIEF DESCRIPTION OF THE FIGURES

The present invention will be better understood from reading the following description of non-limiting embodiments, with reference to the attached drawings, wherein below:

FIG. 1 is a perspective view of an exemplary rigid support structure suitable for use with embodiments of the present invention;

FIG. 2 is a simplified perspective view of an eight panel flexible bioprocessing vessel suitable for use with embodiments of the present invention;

FIG. 3 is a top perspective view of an apparatus for transporting and installing a flexible bioprocessing vessel according to an embodiment of the invention;

FIG. 4 is a bottom perspective view of the apparatus of FIG. 3;

FIG. 5 is a depiction of the apparatus of FIG. 3 with a flexible bioprocessing vessel, the apparatus placed within an exterior container for transportation;

FIG. 6 is a graphical illustration depicting users moving a flexible bioprocessing vessel via an apparatus for transporting and installing a flexible bioprocessing vessel according to an embodiment of the invention;

FIG. 7 is a perspective view of the apparatus of FIG. 3 depicting a front panel detached from a first wing portion and a second wing portion of a base portion of the apparatus as part of an installation process, as well as a portion of a flexible bioprocessing vessel, according to an embodiment of the invention;

FIG. 8 is a perspective view of the apparatus of FIG. 7 depicting tabs with hoist eyes extended for potential use as part of the installation process;

FIG. 9 is a perspective view of the apparatus of FIG. 7 depicting a first wing portion and a second wing portion of the apparatus are relative to a base portion to form a storage cavity for a flexible bioprocessing vessel;

FIG. 10 is a perspective view of the apparatus of FIG. 7 in an installed position;

FIG. 11 is a perspective view of an apparatus for transporting and installing a flexible bioprocessing vessel according to an alternative embodiment of the invention;

FIG. 12 is an exploded perspective view of an apparatus for transporting and installing a flexible bioprocessing vessel according to an alternative embodiment of the invention; and

FIG. 13 is an exploded perspective view of an apparatus for transporting and installing a flexible bioprocessing vessel according to an alternative embodiment of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film.

A “vessel,” as the term is used herein, means a flexible bag, a flexible container, a semi-rigid container, or a rigid container, as the case may be. The term “vessel” as used herein is intended to encompass bioprocessing vessels having a wall or a portion of a wall that is flexible, single-use flexible bags, as well as other containers or conduits commonly used in biological or chemical processing, including, for example, cell culture/purification systems, fermentation systems, mixing systems, media/buffer preparation systems, and filtration/purification systems.

As used herein, the term “bag” means a flexible or semi-rigid vessel used, for example, as a mixer or bioreactor for the contents within.

Embodiments may be utilized in connection with a wide variety of biological and chemical processes, which are referred to generally herein as “bioprocessing.” This term encompasses, but is not limited to, the various processes that occur in bioreactors, mixers, fermenters, and the like. A “bioprocessing vessel” is a vessel suitable for use with or in a bioreactor, mixer, fermenter, or other biological or chemical processing device. Certain embodiments may be suitable for use in other industries where size, ease of installation and use, and efficient versatile mixing of fluids are desirable.

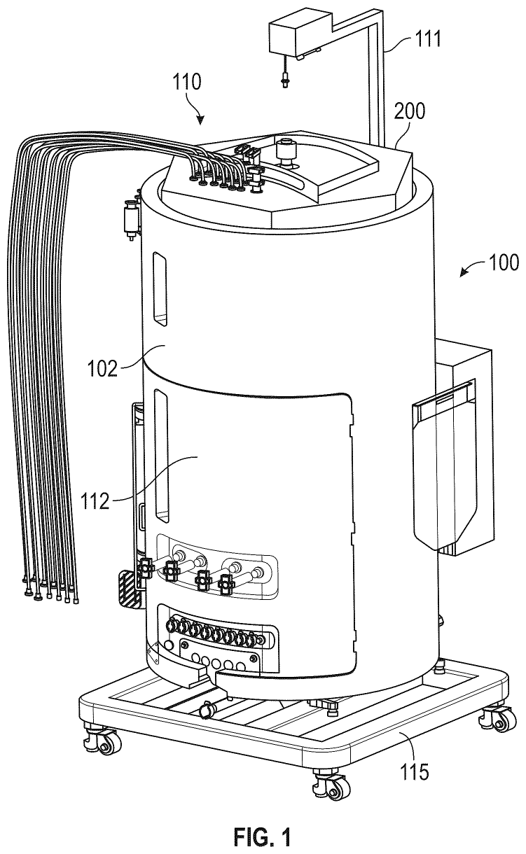

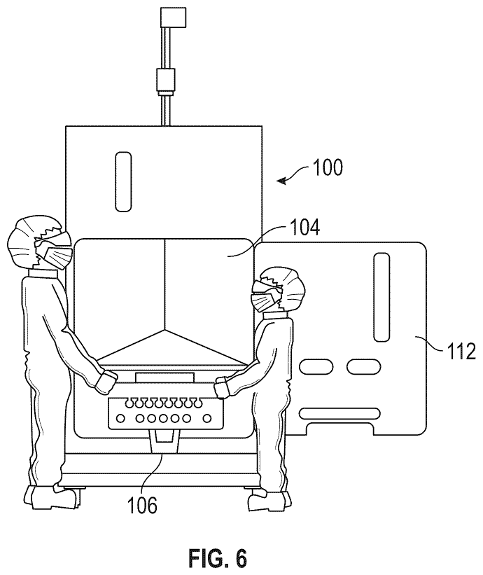

Referring to FIG. 1 and FIG. 6. a tank/rigid support structure 100 suitable for use with embodiments of the invention is depicted. The rigid support structure 100 includes a rigid body 102 having an interior 104 that includes a bottom panel 106. The interior 104, which is cylindrical, has a substantially open top 110 and a selectively openable door 112 which allows access to the interior 104, which is open and configured to receive a flexible bioprocessing vessel 200 (FIG. 3). The rigid support structure 100 further includes a stand portion 115 attached to the rigid body 102 which allows for access to the space below the body.

Although embodiments are described as for use with cylindrical reactor tanks, embodiments may be suitable for use with tanks (and vessels that fit therein) having other geometries, e.g., square, rectangular, and hexagonal.

In embodiments, the rigid support structure 100 may further include a hoist mechanism 111 to mechanically lift a vessel to assist in the installation of the vessel within the structure. As will be appreciated, such a mechanism may be particularly suitable for use with larger vessels and support structure, e.g., 2000 L vessels and larger.

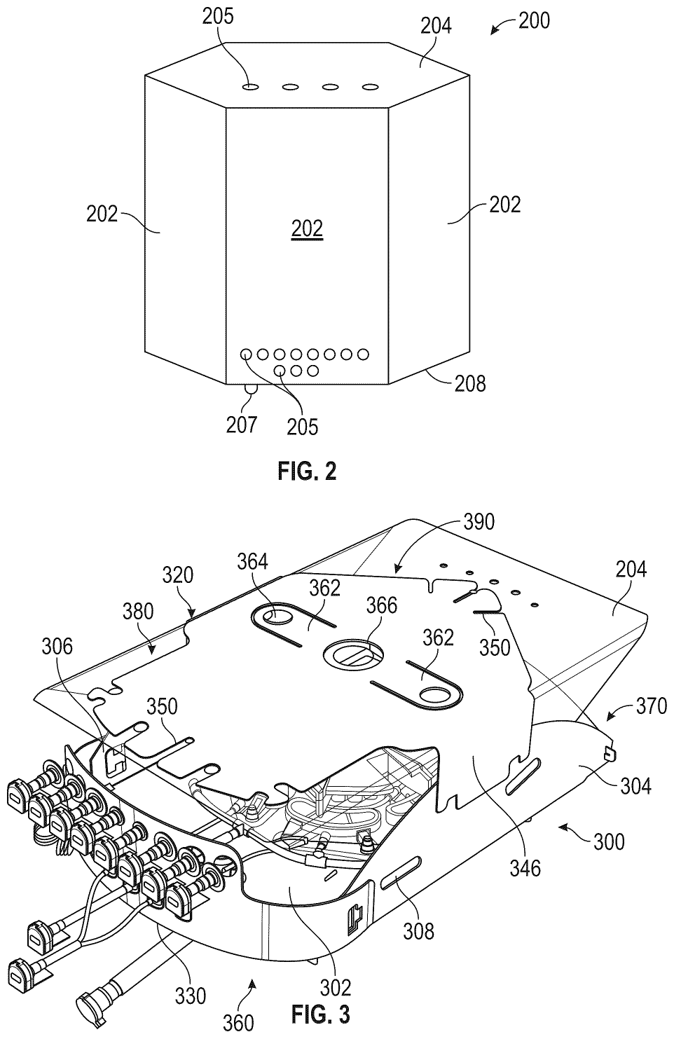

As shown in FIG. 1 and FIG. 2, suitable vessels for use with embodiments of the invention include a flexible bioprocessing vessel 200 that is hexagonal. The flexible bioprocessing vessel 200 includes at least eight flexible panels forming sides, top and bottom of the flexible bioprocessing vessel. The flexible bioprocessing vessel 200 includes six flexible side panels 202, a top panel 204, and a bottom panel 208 located on an opposite end of the hexagonal flexible bioprocessing vessel 200 from the top panel 204. The eight flexible panels are joined together, e.g., welded, to define an interior configured for processing a fluid. In other embodiments, an additional side panel may be included or removed to change the capacity of the vessel.

As shown in simplified schematic form in FIG. 2, the hexagonal flexible bioprocessing vessel 200 further includes a plurality of fluid inputs/connectors/ports 205 for exhaust filtering, sensing, and adding fluids to the interior cavity. In embodiments, the fluid inputs/connectors/ports 205 are located in one or more rows on a front facing flexible panel of the vessel 200, and on the top panel 204. As will be appreciated, the vessel 200 includes at least one fluid output 207 for fluid removal.

Although embodiments of the invention are shown for use with hexagonal bags located within cylindrical support structures, other embodiments may be used with other bag geometries. Such as, for example, octagonal, square, or other geometries.

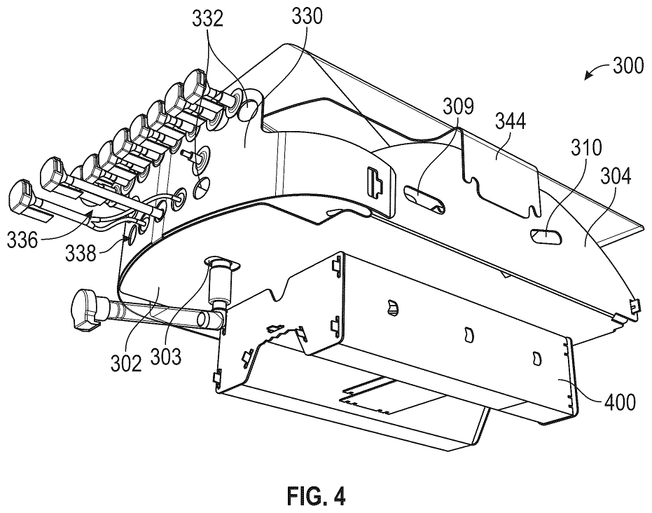

Turning now to FIG. 3 and FIG. 4, an apparatus 300 for transporting and installing a flexible bioprocessing vessel according to an embodiment of the invention is depicted. The illustrated embodiment is configured for attachment to a 2000 L hexagonal flexible bioprocessing vessel. The apparatus 300 includes a base portion 302 having a front end 360 and a rear end 370. The base portion 302 is configured for operative connection to a bottom panel 208, e.g., via direct attachment to the film of the bottom panel or to a vessel port or other component that is welded to the bottom panel, which is hexagonal, of a flexible bioprocessing vessel (FIG. 2). In certain embodiments, the bottom panel may sit on or in the base portion with no direct structural attachment. The base portion 302 further includes a first wing portion 304 and a second wing portion 306, each being hingedly attached to the base portion 302. In use, the first wing portion 304 and the second wing portion 306 are selectively movable relative to the base portion 302 between a first position and a second position.

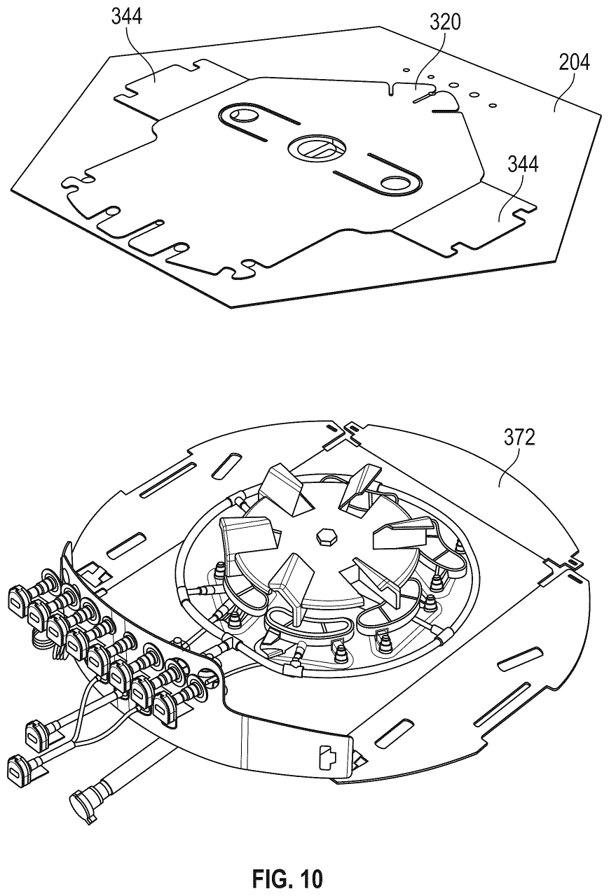

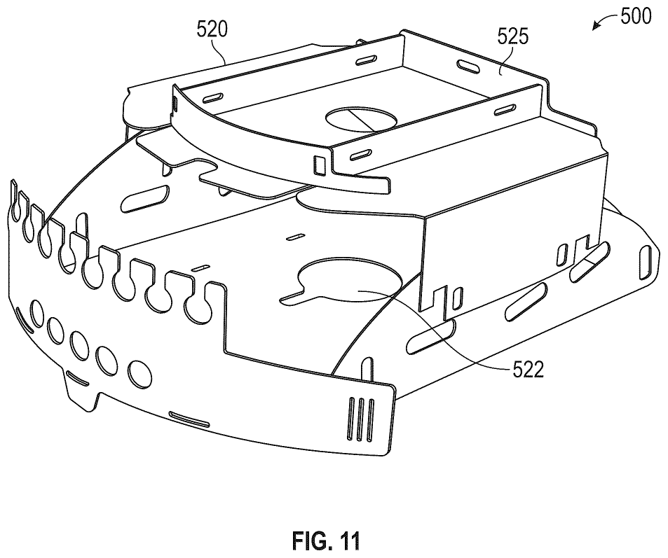

In addition, the base portion 302 includes a rear wing portion 372 (FIG. 10). This portion may be secured to the first and second wing portions via tabs and slots to form a rear wall of the base portion 302. During deployment and use, the rear wing portion 372 may be decoupled from the first and second wing portions so that it lies substantially flat on a bottom surface of a rigid support structure 100.

The base portion 302 may also include a waste line aperture 303 to allow passage of a fluid output/waste drain line from the vessel to an external receptacle. In addition, the base portion 302 includes a large drive motor aperture that allows a drive head of a drive motor to be mated with/connected to an impeller within the vessel.

In embodiments, the first wing portion 304 and the second wing portion 306 each have at least one handle 308. In specific embodiments the first and second wing portions each include two handles, e.g., a first handle 309 and a second handle 310 (FIG. 4). The first handle 309 located proximate to the front end 360 of the apparatus 300 and the second handle 310 located proximate to the rear end 370 of the base portion 302.

Referring to FIG. 3 and FIG. 4, and as discussed in greater detail herein, the at least one handle 308 may be used to pick up the apparatus 300 and vessel 200 and place both within the rigid support structure 100. The at least one handle 308 may also be utilized to remove the apparatus 300 and vessel 200 from external packaging 392 after transport. As will be appreciated, the size, shape, and location of the handles may be selected based on ergonomic factors, i.e., so that a user's hands may extend into the apparatus to facilitate comfortably lifting it. The handles may be spaced apart so that the weight of the apparatus and vessel are balanced.

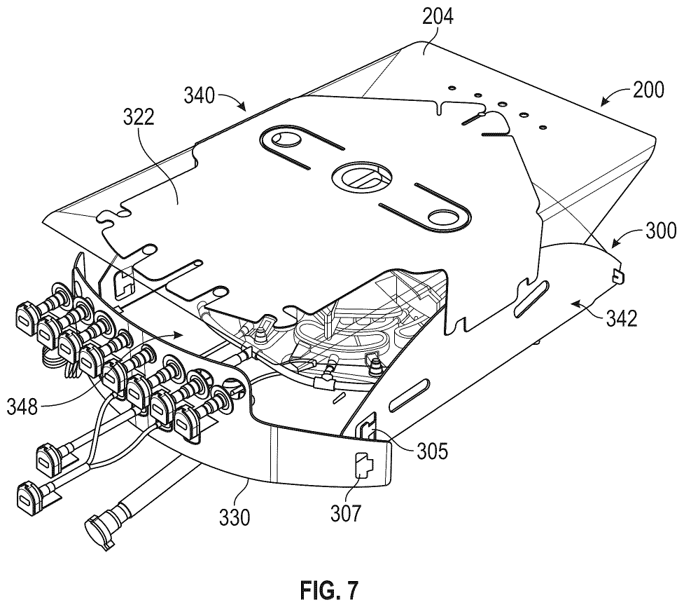

As depicted, the apparatus 300 also includes a front panel 330 attached to the base portion 302. The front panel 330 is connected to the front end 360 of the base portion 302. In embodiments, the front panel 330 includes a plurality of apertures 332 configured to secure and protect tubing, connectors, and the like, extending from the flexible bioprocessing vessel during transportation and/or use. The front panel 330 may be removably attached to the base portion using a variety of fasteners. In the depicted embodiment, the front panel 330 is joined to the base portion via tabs 305 and slots 307 (FIG. 7).

In specific embodiments, there are two rows of apertures that extend across the front panel 330. An upper row 336 and a lower row 338. The placement of the rows and the number of apertures may vary depending upon the placement and number of tubing lines and/or connectors 321 on the flexible bioprocessing vessel 200 that may extend from the vessel. In a specific embodiment, the upper row 336 may include eight apertures and the lower row 338 may include five apertures. Likewise, the size and shape of the apertures may vary as long as they allow for passage of the line/connector/port that is being secured and protected. In embodiments, one or more of the apertures in the upper row may be substantially U-shaped with an open upper portion so that, e.g., a connector may be raised or lowered into the aperture.

In embodiments, the front panel 330 has a curved profile. The curved profile has a radius of curvature that substantially matches a radius of curvature of an interior surface of the rigid support structure, facilitating installation of the vessel within a rigid support structure. The curved profile of the front panel 330 may also match a radius of curvature of the front end 360 of the base portion 302. Indeed, the base portion 302 is substantially circular and configured for placement within a cylindrical rigid support structure.

The apparatus 300 further includes a top plate 320 that is configured for operative connection to a top panel 204 of the flexible bioprocessing vessel 200, e.g., via direct attachment to the film of the top panel or to a vessel port or other component that is welded to the top panel where it remains during use of the vessel 200 for bioprocessing. In certain embodiments, the top plate may sit on the top panel with no direct structural attachment. The top plate 320 includes at least one slot 350 configured to receive and support tubing (e.g., fluid lines) and connectors (e.g., ports or other fittings) facilitating use of the flexible bioprocessing vessel. The at least one slot 350 may be a plurality of slots which may be located on a front surface 380 and/or a rear surface 390 of the top plate 320. In certain embodiments, side surfaces of the top plate may have slots or apertures to support tubing/connectors. Certain embodiments, may have slots on front, rear and side surface so that they may be used with a variety of vessels, e.g., vessels that have exhausts/ports located on front/back/sides of their top panels. As will be appreciated, the slots are sized and shaped to accommodate the structure (e.g., tubing) that is to be placed within the slot. In embodiments, the slots are configured to receive and support tubing and connectors that include exhaust filters.

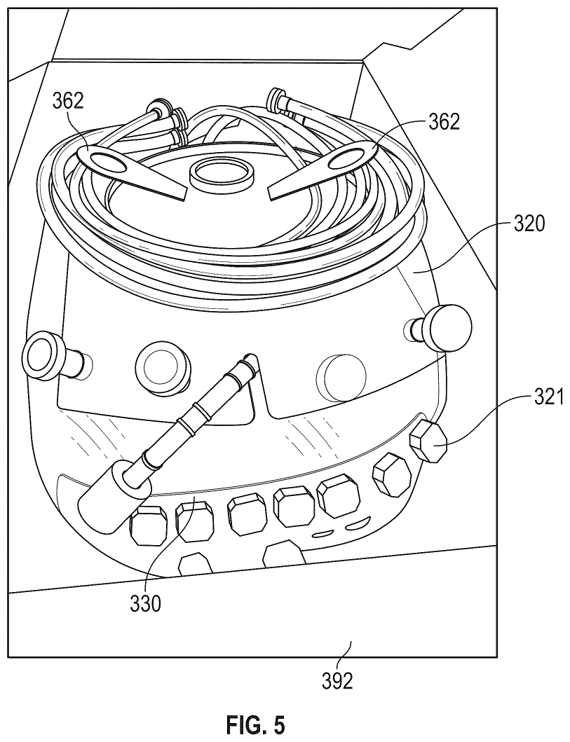

Referring to FIG. 3 and FIG. 5, the top plate 320 further includes one or more tabs 362 that may be folded upward so that tubing shipped with the flexible bioprocessing vessel 200 may be wrapped around the tabs to keep the tubing in place while during shipment/transportation of the vessel. The tabs include a hoist eye 364 configured for connection to a hoist cable to facilitate deployment of the flexible bioprocessing vessel within a rigid support structure. The top plate 320 further includes an attachment mechanism 366 for securing the top plate 320 to the top panel 204 of the vessel.

Referring to FIG. 3, FIG. 4 and FIG. 7, the top plate 320 has a first side 340 and a second side 342. Both the first side 340 and the second side 342 include a flap 344 which, during storage/transportation of the vessel, is folded to that the flaps are substantially perpendicular to an upper surface 322 of the top plate 320. The flaps can be secured to the first wing portion 304 and the second wing portion 306 of the base portion 302 to form a storage cavity 348.

More specifically, in a first position, which is depicted in FIG. 3 and FIG. 4, the first wing portion 304 and the second wing portion 306 are connected to the first side and the second side of the top plate forming a storage cavity for the flexible bioprocessing vessel that facilitates transportation and/or installation of the flexible bioprocessing vessel. In embodiments, and as described in greater detail below, the wing portions are connected to the flaps 344 of the top plate 320.

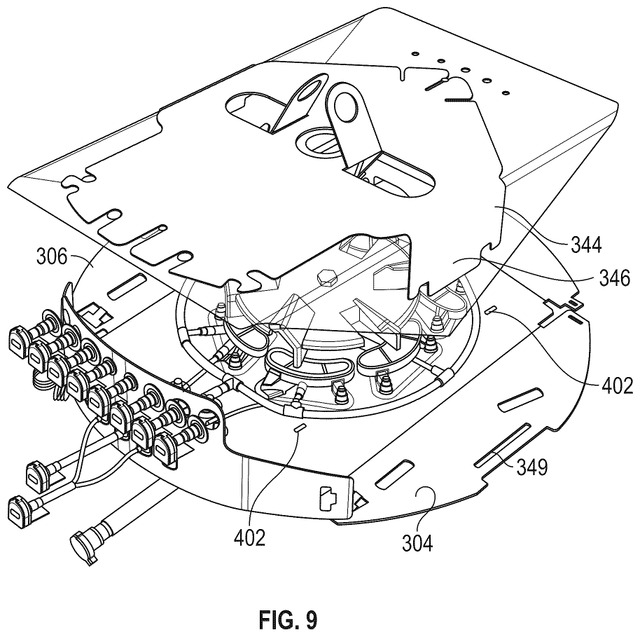

In the second position, which is depicted in FIG. 9, and FIG. 10, the first wing portion 304, the second wing portion 306 are disconnected from the first side 340 and second side 342 of the top plate 320 facilitating installation and/or use of a flexible bioprocessing vessel 200 within a rigid support structure 100. As shown in FIG. 10, in the second position the wing portions and the base portion are substantially planar facilitating install and/or use of the flexible bioprocessing vessel within a rigid support structure 100. As depicted, during installation and when the vessel is deployed, the upper surface 322 of the top plate 320 and the flaps 344 may also be substantially planar.

As shown, each flap 344 of the top plate may include one or more tabs 346 that fit through slots 349 in the opposite wing portion. In embodiments, the one or more tabs 346 may include a hole configured to accept a zip tie or like connector to fix the wing portions and flap 344 in place forming the storage cavity 348. Of course, in other embodiments various mechanisms may be employed to secure the wing portions and side flaps.

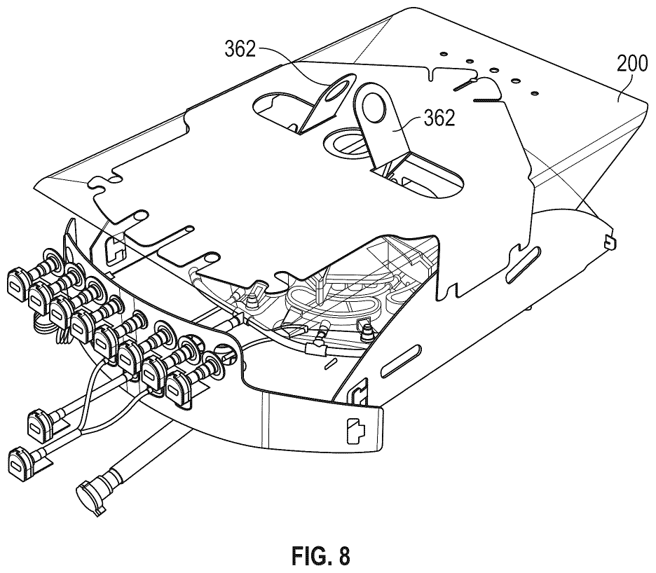

Referring now to FIG. 5, FIG. 6, FIG. 8, FIG. 9, FIG. 10, an installation method is depicted in which two users will initially lift/free the apparatus 300 (which includes the vessel 200 in the storage cavity 348) from external packaging 392. Generally, this will initially involve two users, one on each side of the base portion gripping handles located on the wing portions. The apparatus 300 is then placed in the cylindrical interior of the rigid body of the support structure.

Once the apparatus 300 is in the interior, two users are no longer necessary and one person may continue with the installation. At this point, the front panel 330 is detached from the first wing portion 304 and the second wing portion 306. In an embodiment, the front panel is detached via a tab 305 on each wing portion, each tab removably fitting into a slot 307 on either side of the front panel. The front panel, which is curved, will follow the curvature of a front surface of the rigid support structure/tank. At this stage, each wing portion remains connected to each side of the top plate, e.g., connected to the flaps 344 of the top plate. As will be appreciated, this is facilitated by the plurality of apertures in the front panel.

Referring to FIG. 1 and FIG. 8, in certain embodiments, the tabs 362 located on the top plate 320 may be raised to that a hoist cable may be connected to the hoist eyes 364. As will be appreciated, the hoist cable is connected to a hoist mechanism 111 which assists in deployment of the vessel 200, along with air inflation. In other embodiments, the hoist eyes 364 may not be utilized, and indeed not present, and the vessel 200 may be hoisted into place using a plurality of attachment points on the vessel itself (not shown).

Once the sides of the plate (e.g., the flaps 344) are detached from the first and second wing portions of the base portion, the top plate may be hoisted. More specifically, the tabs 346 on the flaps 344 slide out of the slots 349 in the wing portions. The wing portions then fold open as shown in FIG. 9. As the top plate and vessel are raised, the still folded flaps protect the vessel from, for example, sticking to the interior of the rigid support structure/tank.

The vessel 200 is then raised to a fully deployed state using hoisting and air inflation. Once deployed the flaps 344 of the top plate extend out so that the top plate is substantially planar. This configuration is shown in FIG. 10, although only the top panel 204 of the vessel 200 is depicted. Notably, the top plate remains attached to the vessel during use and may be utilized to uninstall/remove vessel after use.

In an embodiment, the top plate 320 is attached to the vessel via an attachment mechanism 366, e.g., a port welded to the vessel. As will be appreciated, other attachment mechanisms are possible and in certain embodiments, the apparatus 300 may be thermally welded to or otherwise unitary with/permanently attached to the vessel. In a specific embodiment, the top plate 320 is secured to a 2″ inch TC port for a foam camera and to a ¼″ condensate return line.

In embodiments, the apparatus 300 may be manufactured from a rigid or semi-rigid polymeric material capable of holding the weight of the flexible hexagonal vessel without excessively deforming or breaking. The apparatus 300 may be sterilized prior to attachment to the flexible bioprocessing vessel.

In certain embodiments, the first wing portion, second wing portion, and flaps may be hinged/foldable via a section of thinned or perforated material forming fold lines in the apparatus 300. The apparatus 300 may be manufactured via a variety of processes and the invention is not limited in this regard.

As mentioned, embodiments allow for the transportation and installation of hexagonal flexible bioprocessing vessels within cylindrical support structures/tanks. In specific embodiments, the base portion of the apparatus, has a width of about 750 mm cm when configured for use with a 2000 L flexible bioprocessing vessel that is hexagonal. As will be appreciated, however, embodiments the invention are not so limited as long as the width of the apparatus is sufficient to allow for a proper alignment and fit within a rigid cylindrical support structure. In embodiments, the hexagonal flexible bioprocessing vessel may be folded in a specific manner that allows it to fit into the apparatus 300.

Referring back to FIG. 4, embodiments may include at least one selectively removable support base 400, e.g., a tray, configured for connection to an exterior of the base portion to protect, for example, the drain hose and/or a downwardly protruding portion of the impeller seat and provide storage during shipping while the apparatus 300 is in an external packaging/box (FIG. 5). The selectively removable tray may be secured to the base portion via tabs that fit into apertures 402, though the invention is not limited in that regard (FIG. 9). In embodiments, multiple selectively removable trays may be utilized.

Referring to FIG. 11 an alternative embodiment is depicted. In this embodiment, the apparatus 500 includes a top plate 520 that features a raised rectangular structure that is configured to receive and secure tubing during transport. As shown, the apparatus 500 includes a drive motor aperture 522 that is configured to receive a mating portion of the impeller which resides in the vessel/bag allowing the impeller to be operatively connected to a drive head of a drive motor. As will be appreciated, the other embodiments described herein all contain a drive motor aperture.

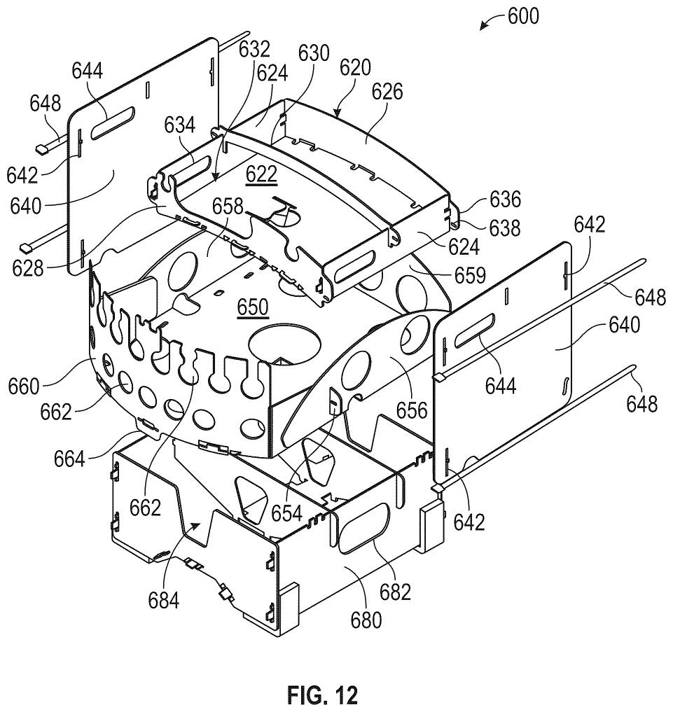

Referring now to FIG. 12 another embodiment of an apparatus 600 for transporting and installing a flexible bioprocessing vessel is depicted. The illustrated embodiment is configured for attachment to a 500 L hexagonal flexible bioprocessing vessel. Similar to the embodiment of FIG. 11, the apparatus 600 features a top plate 620 that includes a raised rectangular structure that is configured to receive and secure tubing during transport. The top plate 620 includes a bottom surface 622, as well as two opposed sides 624, a back 626, and a front 628, which form an interior configured to receive vessel tubing, e.g., coiled tubing, during transportation. The two opposed sides 624. the front 628 and/or the back 626 may be folded into position relative to the bottom surface 622 and joined together via tabs and slots. In other embodiments, the top plate 620 may be molded or otherwise formed of a single piece of material. The top plate 620 may be operatively connected to the flexible bioprocessing vessel via attachment mechanisms described above.

In embodiments, a removable securing beam 630 extends between, for example, the two opposed sides 624. The securing beam 630 may fit into slots formed in the sides to join the same. As will be appreciated, other connection mechanisms may be employed. In embodiments, the removable securing beam 630 is made from the same material as the top plate 620, e.g., a flexible polymeric material.

The front 628 may include one or more apertures, slots and/or openings 632 configured to receive and/or support tubing of the flexible bioprocessing vessel. The two opposed sides 624 may include handles 634.

The apparatus 600 further includes a base portion 650 that is configured for operative connection to a bottom panel 208, e.g., via direct attachment to the film of the bottom panel or to a vessel port or other component that is welded to the bottom panel, which is hexagonal, of a flexible bioprocessing vessel. The base portion 650 further includes a first wing portion 656 and a second wing portion 658, each being hingedly attached to the base portion 650. The base portion 650 may also include a rear flap 659 which is also hingedly attached to the base portion 650.

As with prior embodiments, in use, the first wing portion 656 and the second wing portion 658 are selectively movable relative to the base portion 650 between a first position and a second position. Wherein in the first position, the first wing portion and the second wing portion are operatively connected to a first side and a second side of the top plate forming a storage cavity for the flexible bioprocessing vessel that facilitates transportation and/or installation of the flexible bioprocessing vessel. In the second position, the first wing portion and the second wing portion are disconnected from the first side and second side of the top plate 620 facilitating installation and/or use of the flexible bioprocessing vessel within a rigid support structure. The wing portions and the rear flap, if present, are curved so that, in the second position, they may lay flat and form a substantially round base configured to fit within a cylindrical rigid support structure.

In addition, the base portion 650 includes a large drive motor aperture that allows a drive head of a drive motor to be mated with/connected to an impeller within the vessel.

As depicted, the apparatus 600 also includes a front panel 660 attached to the base portion 650. The front panel 660 is connected to a front end of the base portion 650. In embodiments, the front panel 660 includes a plurality of apertures 662 configured to secure and protect tubing, connectors, and the like, extending from the flexible bioprocessing vessel during transportation and/or use. The front panel 660 may be removably attached to the base portion 650 using a variety of fasteners, e.g., tabs and slots, or may be unitary with the base portion 650.

As shown, the front panel 660 includes a locator tab 664. The locator tab 664 is used to locate/align the apparatus 600 within the rigid support structure during installation. The locator tab 664, which fits into a slot, channel, cut out, or other opening formed in the bottom of the rigid support structure/tank may also prevent or reduce rotation of the flexible bioprocessing vessel during installation/deployment of the same. The front panel 660 is curved to facilitate placement within a cylindrical rigid support structure.

In the depicted embodiment, the apparatus 600 includes removable side panels 640, which may be used to operatively connect the base portion 650 to the top plate 620. The removable side panels 640 may be connected to the base portion 650 and top plate 620 using slots 642, which receive tabs 636 on the top plate 620. The tabs 636 include apertures 638 which are configured to receive a cable tie 648. Likewise, the side panels may be connected to the base portion 650 via tabs 654 which also have apertures configured to receive a cable tie 648. In use, the side panels 640 are attached to the top plate and base portion via four cable ties, with two cable ties per side panel. As will be appreciated, the invention is not limited to securing the side panels via a tab/slot/cable ties configuration and other attachment mechanisms may be employed.

As shown, and as with prior embodiments, the apparatus 600 further includes at least one selectively removable support base 680, configured for connection to an exterior of the base portion 650 to protect, e.g., the drain hose and/or a downwardly protruding portion of the impeller seat and provide storage during shipping while the apparatus 600 is in an external packaging. The selectively removable support base 680 may be secured to the base portion 650 via tabs that fit into apertures or via other mechanisms. The selectively removable support base 680 may include a cut out 682 which may be used to visualize the interior of the support base and its contents. The cut out 682 may also double as a handle. As shown, the interior of the selectively removable support base 680 may be divided into compartments 684.

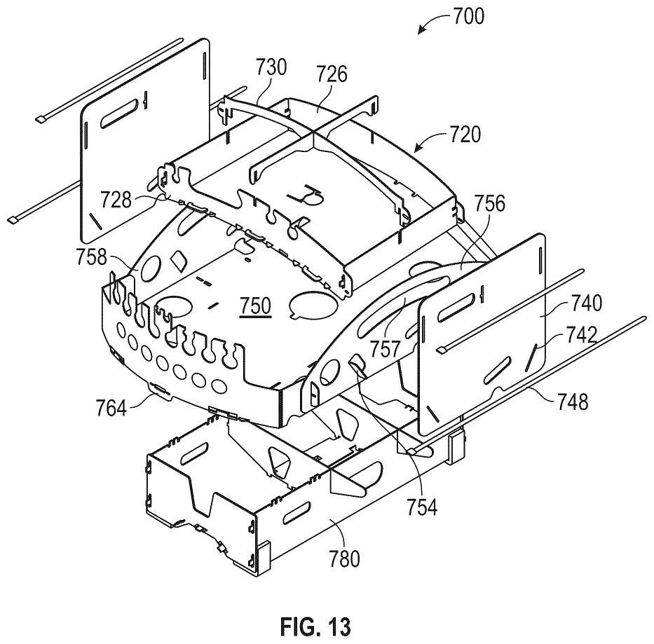

Turning now to FIG. 13, another embodiment of an apparatus 700 for transporting and installing a flexible bioprocessing vessel is depicted. The illustrated embodiment is configured for attachment to a 2000 L hexagonal flexible bioprocessing vessel. As shown, the apparatus 700 is similar to the embodiment depicted in FIG. 12. That is, it includes a top plate 720, side panels 740, base portion 750, and selectively removable support base 780. The side panels 740 include several slots that allow the side panels 740 to be connected to the top plate 720 and base portion 750. These include angled slots 742 that mate with angled tabs 754 on the base portion 750. The base portion 750 includes first and second wing portions 756, 758. The wing portions include handles 757 which facilitate installing a flexible bioprocessing vessel in a rigid support structure. As with prior embodiments, in use, the first wing portion 756 and the second wing portion 758 are selectively movable relative to the base portion 750 between a first position and a second position.

The top plate 720 includes a removable securing beam 730. The beam 730 may fit into slots formed in the sides of the top plate to extend from side to side. The beam 730 further extends between the front 728 and rear 726 of the top plate 720. The beam 730 may be used to secure tubing to the top plate 720.

Embodiments of the invention also contemplate a method for transporting and installing a flexible bioprocessing vessel 200 which includes attaching a base portion 302 of an apparatus 300 to a bottom panel 208 of the flexible bioprocessing vessel 200. The method further includes attaching a top plate 320 of the apparatus 300 to a top panel 204 of the flexible bioprocessing vessel 200, the base portion 302 and top plate 320 remaining attached to the flexible bioprocessing vessel 200 after installation within a rigid support structure 100 and operatively attaching the top plate to the base portion to form a storage cavity 348 for the flexible bioprocessing vessel. The method also includes transporting the flexible bioprocessing vessel 200 to a desired location and installing the flexible bioprocessing vessel 200 with a rigid support structure 100.

In an embodiment, installing the flexible bioprocessing vessel includes placing the support apparatus within a rigid support structure 100, detaching the top plate from the base portion, deploying the flexible bioprocessing vessel.

In an embodiment, the method further includes securing a selectively removable support base 400 to an exterior of the base portion.

In embodiments, the base portion is operatively attached to the top plate via one or more removable side panels.

In an embodiment, the method further includes securing tubing of the flexible bioprocessing vessel within apertures in a front panel of the base portion of the support apparatus prior to transporting the flexible bioprocessing vessel.

In embodiments, the support apparatus may assist in removal of the flexible bioprocessing vessel from the rigid support structure and disposal of said vessel after fluid has been removed from the vessel and processing is complete. That is, users may utilized the handles to remove the support apparatus and vessel from the rigid support structure.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

While the dimensions and types of materials described herein are intended to define the parameters of the invention, they are by no means limiting and are exemplary embodiments. Many other embodiments will be apparent to those of skill in the art upon reviewing the above description.

The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

Moreover, in the following claims, terms such as “first,” “second,” “upper,” “lower,” “bottom,” “top,” etc. are used merely as labels, and are not intended to impose numerical or positional requirements on their objects. Further, the limitations of the following claims are not written in means-plus-function format and are not intended to be interpreted as such, unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal language of the claims.

1. An apparatus for transporting and installing a flexible bioprocessing vessel comprising:
a base portion configured for operative connection to a bottom panel of the flexible bioprocessing vessel, the base portion having a first wing portion and a second wing portion that are selectively movable relative to the base portion between a first position and a second position;
a top plate configured for operative connection to a top panel of the flexible bioprocessing vessel, the top plate remaining attached to the flexible bioprocessing vessel during use;
a front panel connected to a front end of the base portion, the front panel including a plurality of apertures configured to secure and protect tubing and/or connectors extending from the flexible bioprocessing vessel during transportation and/or use; and
wherein in the first position, the first wing portion and the second wing portion are operatively connected to a first side and a second side of the top plate forming a storage cavity for the flexible bioprocessing vessel that facilitates transportation and/or installation of the flexible bioprocessing vessel;
wherein in the second position, the first wing portion and the second wing portion are disconnected from the first side and second side of the top plate facilitating installation and/or use of the flexible bioprocessing vessel within a rigid support structure.

2. The apparatus of claim 1 further comprising:
a selectively removable support base configured for connection to an exterior of the base portion, the selectively removable support base configured to protect portions of the flexible bioprocessing vessel that extend from the apparatus during transportation of the flexible bioprocessing vessel.

3. The apparatus of claim 1 wherein a front surface and/or a rear surface of the top plate includes at least one slot, aperture, and/or opening configured to receive and/or support tubing of the flexible bioprocessing vessel.

4. The apparatus of claim 1 wherein the first wing portion and the second wing portion each have at least one handle.

5. The apparatus of claim 1 wherein the base portion is substantially circular and configured for placement within a cylindrical rigid support structure.

6. The apparatus of claim 1 wherein the front panel has a curved profile.

7. The apparatus of claim 1 wherein the first wing portion and the second wing portion are connected to the first side and the second side of the top plate via removable side panels.

8. The apparatus of claim 7 wherein the removable side panels are secured to the first wing portion and/or the second wing portion by one or more cable ties.

9. The apparatus of claim 8 wherein each of the removable side panels include one or more handles.

10. The apparatus of claim 1 wherein the top plate is configured to receive tubing of the flexible bioprocessing vessel during transportation.

11. The apparatus of claim 10 further comprising:
a removable beam configured to secure the tubing of the flexible bioprocessing vessel to the top plate.

12. The apparatus of claim 1, configured for use with a 2000 L flexible bioprocessing vessel.

13. The apparatus of claim 1, wherein the flexible bioprocessing vessel includes at least 8 panels that are secured together to form an interior configured to receive fluid for bioprocessing.

14. A system for transporting and installing a flexible bioprocessing vessel comprising:
a flexible bioprocessing vessel having a body that includes a plurality of panels that are secured together to form an interior cavity configured to receive fluids for bioprocessing, the body having a bottom panel and a top panel;
a base portion operatively connected to the bottom panel of the flexible bioprocessing vessel;
a top plate operatively connected to the top panel of the flexible bioprocessing vessel, the top plate and base portion remaining attached to the flexible bioprocessing vessel during transportation and/or use;
a front panel operatively connected to a front end of the base portion, the front panel including a plurality of apertures configured to secure and protect tubing and/or connectors extending from the flexible bioprocessing vessel during transportation and/or use; and
wherein in a first position, the base portion is operatively connected to the top plate forming a storage cavity for the flexible bioprocessing vessel facilitating transportation of the flexible bioprocessing vessel;
wherein in a second position, the base portion and the top plate are disconnected from each other facilitating installation and use of the flexible bioprocessing vessel within a rigid support structure.

15. The system of claim 14 further comprising:
a selectively removable support base configured for connection to an exterior of the base portion, the selectively removable support base configured to protect portions of the flexible bioprocessing vessel that extend from the base portion during transportation of the flexible bioprocessing vessel.

16. The system of claim 14 wherein a front surface and/or a rear surface of the top plate includes at least one slot configured to receive and support tubing facilitating use of the flexible bioprocessing vessel.

17. The system of claim 14 wherein the base portion includes at least one handle.

18. The system of claim 14 wherein the base portion is substantially circular and configured for placement within a cylindrical rigid support structure.

19. The system of claim 14 wherein the flexible bioprocessing vessel includes at least 8 panels that are secured together.

20. A method for transporting and installing a flexible bioprocessing vessel comprising:
operatively connecting a base portion of a support apparatus to a bottom panel of the flexible bioprocessing vessel;
operatively connecting a top plate of the support apparatus to a top panel of the flexible bioprocessing vessel, the base portion and top plate remaining attached to the flexible bioprocessing vessel after installation within a rigid support structure;
operatively attaching the top plate to the base portion to form a cavity for the flexible bioprocessing vessel;
transporting the flexible bioprocessing vessel to a desired location; and
installing the flexible bioprocessing vessel with a rigid support structure.

21. The method of claim 20 wherein installing the flexible bioprocessing vessel comprises:
placing the support apparatus within a rigid support structure;
detaching the top plate from the base portion; and
deploying the flexible bioprocessing vessel.

22. The method of claim 20 further comprising:
securing a selectively removable support base to an exterior of the base portion.

23. The method of claim 20 further comprising:
securing tubing of the flexible bioprocessing vessel within apertures in a front panel of the base portion of the support apparatus prior to transporting the flexible bioprocessing vessel.

24. The method of claim 20 wherein the top plate is operatively attached to the base portion via one or more removable side panels.
